(19) United States
(12) Patent Application Publication
(10) Pub. No.: US 2023/0183811 A1
(43) Pub. Date: Jun. 15, 2023

(54) METHODS OF DETECTING THE EFFICACY OF ANTICANCER AGENTS

(71) Applicant: Georgia Tech Research Corporation, Atlanta, GA (US)

(72) Inventors: Costas Arvanitis, Atlanta, GA (US); Anton Bryksin, Atlanta, GA (US); Victor Menezes, Atlanta, GA (US)

(21) Appl. No.: 17/924,765

(22) PCT Filed: May 14, 2021
(86) PCT No.: PCT/US2021/032450

§ 371 Date: Nov. 11, 2022

Related U.S. Application Data
(60) Provisional application No. 63/024,544, filed on May 14, 2020.

(51) Int. Cl. C12Q 1/6886

(52) U.S. Cl. CPC C12Q 1/6886; C12Q 2600/106

(57) ABSTRACT

An exemplary embodiment of the present disclosure provides a method for determining the effectiveness of a pharmaceutical drug in effecting a change in a biological specimen of a patient. The method comprises injecting a fluid into the patient, the fluid comprising a plurality of microbubbles; directing an ultrasound signal to the biological specimen of the patient; and detecting a level of cfDNA molecules in the patient.

### CROSS-REFERENCE TO RELATED APPLICATIONS

This application claims the benefit of U.S. Provisional Application Ser. No. 63/024,544, filed on 14 May 2020, which is incorporated herein by reference in its entirety as if fully set forth below.

### GOVERNMENT LICENSE RIGHTS

This invention was made with government support under Grant No. R00EB016971, awarded by the National Institutes of Health. The government has certain rights in the invention.

### FIELD OF THE DISCLOSURE

The various embodiments of the present disclosure relate generally to detection methods, and more particularly to methods of determining the efficacy of pharmaceutical agents.

### BACKGROUND

The development of noninvasive methods to detect and monitor primary and metastatic brain tumors continues to be a major challenge in oncology. Over the past years the ability of digital polymerase chain reaction-based technologies to detect tumor cell free DNA (cfDNA) fragments from various cancer types (e.g., pancreatic, breast, hepatocellular, etc.) has improved dramatically. Although this technology is used to detect lung cancer without the need for biopsy, its application to brain tumors is still disappointing. It is assumed that this is because the cfDNA fragments, which have sizes ranging from 200 pb˜7.5 kDa, which is well above the cutoff threshold of about 400 Da for passive transport across the blood-brain barrier (BBB) and non-leaky parts of the blood-tumor barrier (BTB), are blocked from entering the circulation in the blood of a patient. This suggests that identifying methods to overcome these barriers may allow to develop sensitive and accurate methods to measure mutant cfDNA from primary and metastatic brain tumors. Such methods can be pertinent to breast cancer patients, as the brain is a sanctuary site, where cancer cells can escape the effects of adjuvant therapy and can thus be the only site of disease recurrence. In addition, there is significant genomic heterogeneity between the primary tumor and brain metastases. Thus, detection of cfDNA shed from the brain metastasis into the circulation can not only provide accurate diagnosis without the need for a biopsy, but also allow the identification of potentially actionable genetic alterations in the metastases, enhancing our ability to deliver personalized medicine. Accordingly, there is a need for improved methods of detecting levels of cfDNA from cancerous tumors in patients to determine the effectiveness of pharmaceutical agents in attacking the tumor cells.

### BRIEF SUMMARY

An exemplary embodiment of the present disclosure provides a method for determining the effectiveness of a pharmaceutical drug in treating a tumor of a patient, comprising. The method comprises injecting a fluid into the patient, the fluid comprising a plurality of microbubbles; directing an ultrasound signal to the tumor of the patient; and detecting a level of cell-free DNA (“cfDNA”) molecules in the patient.

In any of the embodiments disclosed herein, the level of cfDNA molecules in the patient can be indicative of an effectiveness of the pharmaceutical drug in causing cell death in cells of the tumor.

In any of the embodiments disclosed herein, the fluid can be injected into the patient systemically.

In any of the embodiments disclosed herein, the ultrasound signal can mechanically interact with at least a portion of the plurality of microbubbles to permeabilize a blood tumor barrier (“BTB”) of the tumor.

In any of the embodiments disclosed herein, cfDNA molecules can traverse from inside the tumor, through the BTB, and into the blood stream of the patient.

In any of the embodiments disclosed herein, the ultrasound signal can mechanically interact with at least a portion of the plurality of microbubbles to permeabilize a blood brain barrier (“BBB”) of the patient.

In any of the embodiments disclosed herein, cfDNA molecules can traverse from inside the tumor, through the BTB, through the BBB, and into the blood stream of the patient.

In any of the embodiments disclosed herein, detecting the level of cfDNA molecules in the patient can comprise performing a polymerase chain reaction.

In any of the embodiments disclosed herein, the polymerase chain reaction can be a digital polymerase chain reaction.

In any of the embodiments disclosed herein, the cfDNA molecules can be circulating tumor DNA (“ctDNA”) molecules.

In any of the embodiments disclosed herein, detecting the level of cfDNA molecules in the patient can comprise detecting the level of cfDNA molecules in a blood sample of the patient.

In any of the embodiments disclosed herein, detecting the level of cfDNA molecules in a blood sample of the patient can comprise: detecting the level of cfDNA molecules in a first blood sample of the patient, the first blood sample collected from the patient prior to administration of the pharmaceutical drug; detecting the level of cfDNA molecules in a second blood sample of the patient, the second blood sample collected from the patient before administration of the pharmaceutical drug; and detecting the level of cfDNA molecules in a third blood sample of the patient, the third blood sample collected from the patient after administration of the pharmaceutical drug.

In any of the embodiments disclosed herein, the method can further comprise comparing the levels of cfDNA molecules in the first, second, and third blood samples.

In any of the embodiments disclosed herein, an increase in the level of cfDNA molecules between the first, second, and third samples can be indicative of an effectiveness of the pharmaceutical agent.

Another embodiment of the present disclosure provides a method for determining the effectiveness of a pharmaceutical drug in effecting a change in a biological specimen of a patient. The method comprises injecting a fluid into the patient, the fluid comprising a plurality of microbubbles; directing an ultrasound signal to the biological specimen of the patient; and detecting a level of cfDNA molecules in the patient.

In any of the embodiments disclosed herein, the level of cfDNA molecules in the patient can be indicative of an effectiveness of the pharmaceutical drug in causing cell death in cells of the biological specimen.

In any of the embodiments disclosed herein, the fluid can be injected into the patient systemically.

In any of the embodiments disclosed herein, the ultrasound signal can mechanically interact with at least a portion of the plurality of microbubbles to permeabilize a blood specimen barrier (“BSB”) of the specimen.

In any of the embodiments disclosed herein, cfDNA molecules can traverse from inside the specimen, through the BSB, and into the blood stream of the patient.

In any of the embodiments disclosed herein, the specimen can be a tumor.

In any of the embodiments disclosed herein, the ultrasound signal can mechanically interact with at least a portion of the plurality of microbubbles to permeabilize a BBB of the patient.

In any of the embodiments disclosed herein, cfDNA molecules can traverse from inside the tumor, through the BSB, through the BBB, and into the blood stream of the patient.

These and other aspects of the present disclosure are described in the Detailed Description below. Other aspects and features of embodiments will become apparent to those of ordinary skill in the art upon reviewing the following description of specific, exemplary embodiments. While features of the present disclosure may be discussed relative to certain embodiments, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

A conventional pharmaceutical therapy for treating cancerous tumors involves administering a pharmaceutical agent to a patient for the purpose of attacking cells located in the tumor. As a result of the death of these cells, cfDNA fragments of these cells are generated in the tumor. Thus, the more effective a pharmaceutical agent or other therapy is in killing the tumor cells, the higher the level of cfDNA fragments. These cfDNA fragments can range from 200 pb˜7.5 kDa in size, which is well above the cutoff threshold of about 400 Da for passive transport across the blood-brain barrier (BBB) and non-leaky parts of the blood-tumor barrier (BTB). Thus, the cfDNA fragments from dead tumor cells may not make their way into the blood stream of a patient, where the levels of those cfDNA fragments could be detected. Accordingly, because the cfDNA fragments do not traverse the BBB or BTB into the blood stream where they can be detected, it is very difficult with current technologies to determine the effectiveness of a treatment for targeting the tumor cells.

A promising minimally invasive approach to disrupt cellular and vascular barriers is focused ultrasound combined with intravenously administered microbubbles. This method utilizes the mechanical interactions between microbubbles oscillating in the ultrasound field and cells, leading to transient formation of nanoscale pores that result in elevated transmembrane transport of molecules. In the brain, these interactions can also lead to the transient disassembly of tight junction complexes and the induction of active transport across the BBB. In the context of cancer therapy, these interactions can allow for the targeted release and delivery of potent anticancer agents to the tumor cells. The present disclosure, however, makes further use of pathways to allow for the transmission of the cfDNA fragments, such as circulating tumor DNA (ctDNA), out of the tumor and into the blood stream where levels of those fragments can be detected to determine the efficacy of the anticancer agents.

An exemplary embodiment of the present disclosure provides a method for determining the effectiveness of a pharmaceutical drug in effecting a change in a biological specimen of a patient. The biological specimen can be many biological specimens, such as various types of cancerous tumors or the brain of the patient. The method can begin after the patient has been previously treated with a pharmaceutical drug, such as an anticancer agent, intended to target (e.g., kill) particular cancer cells. In particular, the method can be used to determine the effectiveness of the pharmaceutical drug in achieving the objective of cancer cell death. For example, in some embodiments, the specimen can be the brain including breast cancer cells.

The method comprises introducing a plurality of microbubbles into the blood stream of a patient. In some embodiments, the microbubbles are introduced into a patient using a fluid comprising the plurality of microbubbles. The fluid and microbubbles can form a solution. The fluid can be injected into the patient many different ways known in the art. In some embodiments, the fluid can be injected into the blood stream of the patient intravenously (IV injection). This can be similar to the way anticancer agents, e.g., pharmaceutical agents, are often injected into a patient. The fluid can be injected into the patient systemically so that the plurality of microbubbles circulate in the bloodstream of the patient traveling throughout the body of the patient.

The method can further comprise directing an ultrasound signal to the biological specimen of the patient. In particular, the ultrasound signal can be directed to the biological specimen (e.g., to a tumor or through the skull to the brain) when the plurality of microbubbles are circulating in the bloodstream of the patient near a barrier between the specimen and the blood of the patient, i.e., a blood-specimen-barrier (BSB), such as the blood-tumor-barrier (BTB) or blood-brain-barrier (BBB). The ultrasound signal can mechanically interact with at least a portion of the plurality of microbubbles to permeabilize the BSB of the specimen. These interactions can form nanoscale pores in the BSB that result in elevated transmembrane transport of molecules across the BSB. When the specimen is the brain, these interactions can result in the transient disassembly of tight junction complexes and the induction of active transport of molecules across the BBB. Thus, in some embodiments, the cfDNA molecules (such as ctDNA molecules) can traverse from inside the specimen, through the BSB (and/or BTB and/or BBB), and into the blood stream of the patient.

The method can further comprise detecting a level of cfDNA molecules in the bloodstream of a patient. In some embodiments, detecting the level of cfDNA molecules in the patient comprises detecting the level of cfDNA molecules in a blood sample of the patient. Blood samples can be taken at different points of time and the level of cfDNA molecules in that blood sample determined to assess the effectiveness of the pharmaceutical drug in causing cell death in the specimen of interest, e.g., tumor. For example, in some embodiments, blood samples are collected before, during, and after administration of the pharmaceutical drug to the patient. The levels of cfDNA molecules in each of these samples can be measured and compared. If the level of cfDNA molecules increases from blood samples taken before to blood samples taken during and after administration of the pharmaceutical drug, that is indicative that the pharmaceutical drug was effective in causing cell death of the specimen.

The level of cfDNA molecules in the bloodstream of a patient can be indicative of an effectiveness of the pharmaceutical drug in causing cell death in cells of the biological specimen. In any of the embodiments disclosed herein, detecting the level of cfDNA molecules in the patient can comprise performing a polymerase chain reaction, such as a digital polymerase chain reaction.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A method comprising:
injecting a fluid comprising microbubbles into a patient being treated by a pharmaceutical drug; and
detecting a level of cell-free DNA (“cfDNA”) molecules in the patient through analyses of an ultrasound signal having been directed toward a biological specimen of the patient.

2. The method of claim 1, wherein the biological specimen is a tumor; and
wherein the level of cfDNA molecules in the patient is indicative of an effectiveness of the pharmaceutical drug in causing cell death in cells of the tumor.

3. The method of claim 2, wherein injecting comprises systematically injecting the fluid into the patient.

4. The method of claim 2 further comprising directing the ultrasound signal;
wherein the level of cfDNA molecules in the patient is indicative of an effectiveness of the pharmaceutical drug in causing cell death in cells of the tumor; and
wherein the ultrasound signal mechanically interacts with at least a portion of the microbubbles to permeabilize a blood tumor barrier (“BTB”) of the tumor.

5. The method of claim 4, wherein cfDNA molecules traverse from inside the tumor, through transiently formed nanoscale pores in the BTB via the mechanical interaction, and into the blood stream of the patient.

6. The method of claim 4, wherein the ultrasound signal mechanically interacts with at least a portion of the microbubbles to further permeabilize a blood brain barrier (“BBB”) of the patient.

7. The method of claim 6, wherein cfDNA molecules traverse from inside the tumor, through the BTB, through transiently formed nanoscale pores in the BBB, and into the blood stream of the patient.

8. The method of claim 2, wherein detecting the level of cfDNA molecules in the patient comprises performing a polymerase chain reaction.

9. The method of claim 8, wherein the polymerase chain reaction is a digital polymerase chain reaction.

10. The method of claim 2, wherein detecting the level of cfDNA molecules in the patient comprises detecting the level of cfDNA molecules in a blood sample of the patient.

11. A method comprising:
detecting a level of cell-free DNA (“cfDNA”) molecules secreted by a biological specimen in a first blood sample of a patient, the cfDNA secreted by the biological specimen in the patient, and the first blood sample collected from the patient prior to administration of a pharmaceutical drug potentially effecting a change in the biological specimen;
detecting the level of cfDNA molecules in a second blood sample of the patient, the second blood sample collected from the patient before administration of the pharmaceutical drug; and
detecting the level of cfDNA molecules in a third blood sample of the patient, the third blood sample collected from the patient after administration of the pharmaceutical drug.

12. The method of claim 11 further comprising comparing the levels of cfDNA molecules in the first, second, and third blood samples;
wherein the level of cfDNA molecules in a third blood sample are resultant from an ultrasound signal mechanically interacting with microbubbles in proximity to the biological specimen that permeabilizes a blood tumor barrier (“BTB”) of the biological specimen.

13. The method of claim 12, wherein an increase in the level of cfDNA molecules between the first, second, and third samples is indicative of an effectiveness of the pharmaceutical agent.

14. The method of claim 13, wherein the cfDNA molecules are circulating tumor DNA (“ctDNA”) molecules.

15. The method of claim 1, wherein:
the biological specimen is a tumor and the method is a method for determining the effectiveness of the pharmaceutical drug in treating the tumor; or
the method is for determining the effectiveness of the pharmaceutical drug in effecting a change in the biological specimen.

16. The method of claim 15, wherein the level of cfDNA molecules in the patient is indicative of an effectiveness of the pharmaceutical drug in causing cell death in cells of the biological specimen.

17. The method of claim 15, wherein the fluid is injected into the patient systemically.

18. The method of claim 15 further comprising directing the ultrasound signal to the biological specimen;
wherein the ultrasound signal mechanically interacts with at least a portion of the microbubbles to permeabilize a blood specimen barrier (“BSB”) of the biological specimen.

19.-24. (canceled)

25. The method of claim 15, wherein detecting the level of cfDNA molecules in the patient comprises:
detecting the level of cfDNA molecules in a first blood sample of the patient, the first blood sample collected from the patient prior to administration of the pharmaceutical drug;
detecting the level of cfDNA molecules in a second blood sample of the patient, the second blood sample collected from the patient before administration of the pharmaceutical drug; and
detecting the level of cfDNA molecules in a third blood sample of the patient, the third blood sample collected from the patient after administration of the pharmaceutical drug.

26. The method of claim 25 further comprising comparing the levels of cfDNA molecules in the first, second, and third blood samples.

27. The method of claim 26, wherein an increase in the level of cfDNA molecules between the first, second, and third samples is indicative of an effectiveness of the pharmaceutical agent.

28. The method of claim 26, wherein the cfDNA molecules are circulating tumor DNA (“ctDNA”) molecules.
